(19) United States
(12) Patent Application Publication
(10) Pub. No.: US 2026/0294284 A1
(43) Pub. Date: Oct. 1, 2026

(54) NOVEL HEARING TEST SYSTEM

(71) Applicant: MELISONO AB, Lund (SE)

(72) Inventor: Bernt BÖHMER, HÖÖR (SE)

(21) Appl. No.: 19/476,030

(22) PCT Filed: May 2, 2024
(86) PCT No.: PCT/SE2024/050419

§ 371 Date: Oct. 16, 2025

(30) Foreign Application Priority Data
May 8, 2023 (SE) 2350563-9

(51) Int. Cl. A61B 5/12; A61B 5/00

(52) U.S. Cl. CPC A61B 5/123; A61B 5/725; A61B 5/742; A61B 5/7475

(57) ABSTRACT

The present invention discloses a software unit intended for hearing assessment, said software unit being arranged for performing a method comprising the steps of:—performing a reference frequency hearing test to establish a reference frequency, for each ear of a test object; and—performing an equal loudness hearing test for each ear, wherein the step of performing a reference frequency hearing test involves starting with a first frequency positioned within a given frequency range, providing a sound with a set sound level choosing a lowest test sound level in a range of from −10 to 50 Phon, to the test object to obtain a hearing response or not, changing the sound level with the first frequency, if the test object provides a hearing response at the chosen lowest test sound level in the range of from −10 to 50 Phon, then choosing the first frequency as the reference frequency, otherwise choosing another frequency within the given frequency range and repeating an iterative procedure with new frequencies within the given frequency range until the software unit detects a frequency X for which the test object provides a hearing response at the chosen lowest test sound level in the range of from −10 to 50 Phon or which is the frequency with the lowest sound level hearing response from the test object for the set frequencies to be tested, and then choosing that frequency X as the reference frequency.

### INTRODUCTION

The commonly used ISO standard hearing threshold test only involves detecting a person's hearing threshold at a handful of frequencies, normally about 6 to 10. The result of the test is recorded in an Audiogram. Whilst the test is useful for detecting a person's hearing loss through the uncovering of an elevated hearing threshold it is not revealing any information about hearing loss above the hearing threshold. Any hearing restoration that is applied based on the test must rely on the assumption that hearing loss is linear at higher sound pressure levels and more or less equal to the elevated threshold. The presented novel Equal Loudness Hearing Test clearly reveals that this is not the case. The hearing ability of a person experiencing hearing loss changes significantly at sound pressure levels above the hearing threshold.

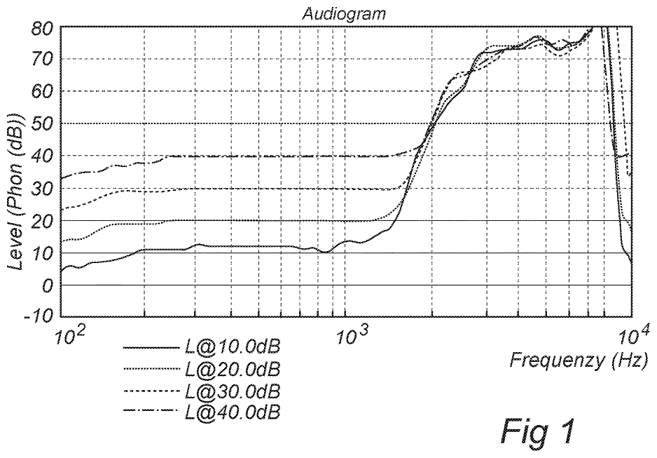

FIG. 1 shows Equal Loudness Hearing Test data at levels 10 Phon, 20 Phon, 30 Phon and 40 Phon for a person with quite significant high frequency hearing loss. The bottom trace shows the lowest 10 Phon level which is just above or at the test subject's hearing threshold at 800 Hz. The traces display to the right a significant elevation of the hearing threshold starting just above 1 kHz. To the left in the diagram, at lower frequencies, the traces are spaced by the expected 10 Phon. At higher frequencies beginning from approximately 2 kHz and above, the spacing is significantly less than 10 Phon. At 300 Hz the 10-40 Phon traces are separated by almost 40 dB, which corresponds to the sound level increase. The slight elevation of the 10 Phon trace between 100 Hz-1 kHz produces a slightly smaller separation to the 20 Phon trace than between the other traces. This is caused by a slight elevation of the hearing threshold influencing at the lowest 10 Phon level. The elevation disappears at 20 Phon and above where the spacing is exactly 10 Phon apart from a few small local deviations. At higher frequencies above 2 kHz where there is significant elevation of the hearing threshold the 10-40 Phon traces are separated by just a few Phon. This reveals that at say 3 kHz the sound pressure level just needs to increase by a couple of Phon to be perceived by the test subject to have increased by 40 Phon. Very clearly the brain starts to radically compensate for the elevated hearing threshold once the sound pressure level is above the hearing threshold and the ear/brain is no longer behaving as a linear device, clearly invalidating the conventionally accepted assumption that hearing loss is linear.

This hearing loss behavior will create a massive problem if the elevated hearing threshold were to be compensated by the present standard methods relying on the assumption of linear hearing behavior. Using standard compensation practice, applying amplification of approximately half to two thirds of the measured elevated hearing threshold, the sound would at lower sound levels and frequencies above approximately 2 kHz evidently not be amplified enough and at higher levels amplified far too much. This is further evidenced by the fact that the test subject whose hearing is measured with ELHT and shown in FIG. 1 has got professionally fitted hearing aids but is not using them since they do not provide any benefit.

Normally the dynamic range compression at frequencies with an elevated hearing threshold is smaller than the case presented in FIG. 1, but all measured test subjects show similar dynamic compression at frequencies where there is an elevation of the hearing threshold. Looking at our collected measurement data it is obvious that the brain automatically compensates for an elevated hearing threshold at higher sound pressure levels when the sound is above the hearing threshold and consequently can be heard. The brain's compensation can be quite dramatic as in FIG. 1 or smaller, but it is always present to a significant degree.

To be able to not only assess whether a hearing loss is present but also to acquire data to enable a proper hearing restoration an apparatus containing software facilitating a new test method is required. For that purpose, the novel Equal Loudness Hearing Test (ELHT) applies several additional test steps compared to the standard ISO hearing threshold test. The following descriptions applies equally to an apparatus, software and method for performing an ELHT.

Apart from the most important aspect, enabling a proper hearing restoration, the ELHT also utilizes available dynamic range in an efficient manner. The current practice, ISO standard hearing test and compensation based on static amplification, consumes a significant portion of the available dynamic range without any benefit. The static amplification approach is in fact exposing the user for a risk of acquiring additional hearing loss due to the disadvantageous and unnecessary exposure to sound pressure levels higher than required. If the standard test reveals an elevated hearing threshold of approximately 70 dB at 3 kHz, as is the case for the person with hearing loss according to FIG. 1, a normal approach would be to amplify sounds at that frequency by about halt to two thirds of the measured elevation. In this case it would be about 35 dB to 45 dB, let us say 40 dB is used.

Shown by ELHT test results, 40 dB amplification is not going to be enough at sound levels below 30 Phon, it is exactly the right amplification for sounds at 30 Phon but at higher sound levels it is too much. At approximately 70 Phon, apparent by the ELHT data in FIG. 1, no amplification is required anymore which makes the static 40 dB amplification incredibly excessive. It is thus no mystery why the person whose hearing is shown in FIG. 1 is not helped by the traditional approach.

With the ELHT approach together with dynamic amplifications that changes depending on sound level the user would not be subjected to any additional amplification at sound levels above 70 Phon which eliminates the risk of sustaining more hearing loss due to high sound levels inadvertently and unnecessarily being generated by the hearing loss compensation.

Regarding utilization of available dynamic range; as an example, a personal sound application in a mobile device with Bluetooth connected earbuds or headphones has already a quite limited dynamic range, limited both by headphone hardware and the Bluetooth connection. The digital lower limit is never better than 16 bit resolution i.e., 96 dB dynamic range and in practice together with hardware limitations it will be less. If 40 dB of that dynamic range is already used by static amplification, we only have at best 56 dB left. If we assume that the maximum output of the Bluetooth headphone hardware is 100 dB we would not be able to reproduce any sounds below 44 dB, which is a hard digital resolution floor not a noise floor. Literally no sounds softer than 44 dB can be reproduced, which means that quite a lot of softer sounds would be totally inaudible and defeat the purpose of the system i.e., restoring hearing for better intelligibility. The same situation applies in hearing aids, regularly operating with more limited bit depths.

### SUMMARY OF THE INVENTION

The basis for the ELHT is perceived equal loudness. The ISO226 standard provides equal loudness information at different sound levels and frequencies which are used by the ELHT to assess hearing loss. The output from the ELHT to a real time signal processing apparatus is gain table data. A real time signal processing apparatus should include correction peaking filters at each frequency that needs to be amplified to restore hearing. The correction peaking filters should dynamically change boost at the filter frequency dependent the on momentary sound pressure at the filter frequency. The gain table data output from the ELHT contains correction gain information at all practical sound pressures for each frequency.

The focus of the method according to the present invention is to establish at least one reference frequency for each ear. According to the most general aspect, the present invention is directed to a software unit intended for hearing assessment, said software unit being arranged for performing a method comprising the steps of:
performing a reference frequency hearing test to establish a reference frequency, for each ear of a test object; and
performing an equal loudness hearing test for each ear, wherein the step of performing a reference frequency hearing test involves starting with a first frequency positioned within a given frequency range, providing a sound, with a set sound level choosing a lowest test sound level in a range of from −10 to 50 Phon, to the test object to obtain a hearing response or not, changing the sound level with the first frequency, if the test object provides a hearing response at the chosen lowest test sound level in the range of from −10 to 50 Phon, then choosing the first frequency as the reference frequency, otherwise choosing another frequency within the given frequency range and repeating an iterative procedure with new frequencies within the given frequency range until the software unit detects a frequency X for which the test object provides a hearing response at the chosen lowest test sound level in the range of from −10 to 50 Phon or which is the frequency with the lowest sound level hearing response from the test object for the set frequencies to be tested, and then choosing that frequency X as the reference frequency.

It should be noted that the present invention is directed to a software unit intended for hearing assessment, but may also provide correction for one or more hearing impairments.

The first ELHT step aims to establish the reference frequencies for the equal loudness evaluation. One reference frequency for each ear is required. The frequency could be the same for both left and right ear but in some cases, it is beneficial to use two different frequencies. The perceived loudness of the reference frequencies at different sound levels is used to balance the perceived loudness of all other frequencies in the test.

The second ELHT step balances perceived loudness of the reference frequencies between the left and right ear. Since all measurements are balanced against the reference frequency's sensed sound level it is vital that the level in the left and right ears are perceived to be equal at all sound pressure levels. If they differ, the whole spatial perception of sound will be skewed, with a degraded spatial hearing ability as a result.

This second step is not mandatory, but is preferred to perform according to the present invention.

The third ELHT step is the equal loudness test. The reference frequency's perceived loudness, one frequency for each ear, is compared with a number of test frequencies suitably spread across the frequency range at a number of appropriate sound pressure levels above the hearing threshold.

In relation to the fourth, fifth and sixth steps described below it should be noted that these are optional according to the present invention. Again, the focus of the method according to the present invention is the establishment of at least one reference frequency for each ear.

The fourth ELHT step carries out the transposition of equal loudness data measured in Phon to sound pressure levels (SPL). Output from the third ELHT step is in Phon. SPL data is required for efficient signal processing in hearing restoration devices like ear buds and hearing aids. SPL is a physical unit normally measured in dBSPL, which is a logarithmic unit using a reference level of 20 micro pascals of sound pressure, whereas Phon is a logarithmic perceptual unit. Perceptual levels measured in Phon are based on human perception of the loudness of sound.

The fifth ELHT data post processing step at first adds missing hearing correction data. The measurement carried out in step 3 only covers a limited dynamic range usually, 10 Phon to 60 Phon. Data for the whole relevant 0 dBSPL to 110 dBSPL dynamic range is required for proper hearing correction. Correction gain data for the missing portions of the dynamic range is calculated by polynomial extrapolation of the available measured range at all frequencies. Next action involves selecting suitable correction frequencies where correction gain should be applied. Frequencies where there is no correction gain required according to the measurement are discarded. Frequencies where the hearing loss is too severe for correction to be feasible are also discarded. This typically happens at the highest frequencies, 9600 Hz and 12800 Hz, where age related hearing loss can make it impossible to hear the frequencies thus making correction pointless and even potentially harmful if too much gain is applied. Finally, the remaining correction gain data is analyzed to find peaks in the hearing loss data where it is suitable to apply correction. One peak frequency can usually cover a frequency range, due to the correction peaking filter bandwidth, which often makes it unnecessary to boost adjacent frequencies closest to the center frequency. When appropriate frequencies are selected two final steps comprising the limiting of maximum gain to avoid acoustical feedback in a hearing correction apparatus and the limiting of gain and thereby maximum sound output at the highest sound pressures.

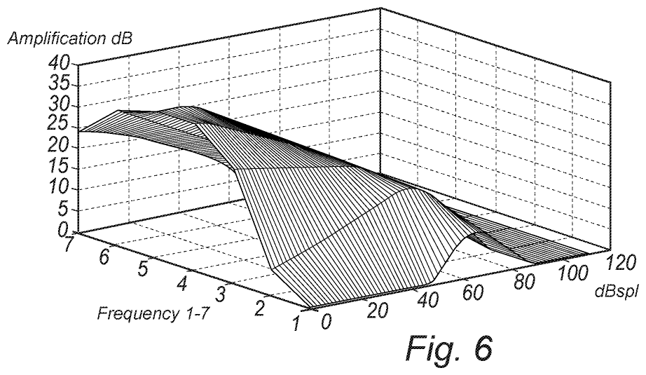

The sixth ELHT step adjusts for aggregate amplification. Amplification from adjacent frequencies interact and add together creating additional gain. Correction filter bandwidths and thereby amplification bandwidths must be wide to achieve good time domain response with limited energy dispersion i.e., ringing. The wide bandwidth consequently causes an overlap between adjacent adjustment frequency bands. To correct aggregate gain to equal the measured required gain an optimization must be performed. The optimization is called gain sail optimization since the applied gain looks like a sail in a three-dimensional plot with frequency, sound level and amplification on the axes, see FIG. 6.

### DETAILED DESCRIPTION OF THE INVENTION

The ELHT System uses a novel hearing test that assesses hearing loss across a broad dynamic range which is significantly different from the present ISO standard hearing threshold test that only provides information about the hearing threshold. The ELHT is based on perceived Equal Loudness of a reference sound compared to a test sound. The test sound level is adjusted until the test person perceives it to be equal to the reference sound in loudness. Typically, the reference sound and test sound are alternatingly played back at an alternation frequency of about 0.5 Hz. The alternation frequency can obviously be changed both to a higher or a lower frequency. A feasible range would be from 0.25 Hz to 1 Hz. Alternation between the two compared tones could be automatic as well as under manual control. The equal loudness levels used are based on the ISO226 standard's levels at different frequencies and sound pressure. Test and reference sound can be pure sine tones, or they could be other types of sound, bandwidth limited to a desired test frequency band. Complex test signals with a wider frequency spectrum than a pure sine can provide more information about hearing ability within the test frequency band and are particularly useful in tests with fewer test frequency bands.

The commonly performed hearing threshold test uses pure sine tones, which can be a significant problem for individuals suffering from tinnitus. With tinnitus it can be quite difficult to distinguish between a perceived tinnitus tone and the test tones generated to assess hearing ability, creating confusion and inaccurate test results.

To improve accuracy and user-friendliness warble tones are used in the ELHT, which is an advantageous alternative to pure sine tones. Many other types of multi frequency tones and noise can of course be used but the warble tone has got several beneficial properties. First it is not possible to confuse a warble tone with tinnitus. Warble tones sound significantly different than the pure sine tones experienced by people experiencing tinnitus. Warble tones have also been scientifically assessed to produce very similar hearing threshold test results compared to pure sine tones. Warble tones have a bandwidth extending just above and below the test frequency and are therefore to some extent less sensitive to local dips and peaks in the frequency response produced by the test equipment and the user's ear and ear canal. This can be particularly relevant at higher frequencies when peaks and dips more easily occur. Lastly, particular for the equal loudness test, it is easier to determine the loudness of a warble tone than it is for a pure sine, making equal loudness comparisons easier. A suitable range for warble tone modulation is 1% to 10%, ELHT typically use 5%. Standard ELHT modulation frequency is 15 Hz with a useful range from 5 Hz to 30 Hz.

The Equal Loudness test results shown in FIG. 1 uses 81 frequencies in the range between 100 Hz and 10 kHz. This many test frequencies are not required for the ELHT. The number of frequencies can be anything between 1 up to the presented 81 or even more. Preferably test frequencies should be 100 Hz, 200Hz, 400Hz, 800Hz, 1200 Hz, 1600 Hz, 2400 Hz, 3200 Hz, 4800 Hz, 6800 Hz, 9600 Hz and 12800 Hz. These frequencies are evenly spread across the frequency range that is relevant for hearing restoration. They are more closely spaced at higher frequencies where higher resolution is required for optimal correction.

The ELHT is performed at sound pressure level intervals of 10 Phon where the lowest test level should be just above the test subject's hearing threshold at the chosen reference frequency. Any other sound pressure level granularity can be used but more closely spaced than 10 Phon provides little benefit in accuracy and is significantly more arduous for the test subject. A wider spacing is occasionally used during the ELHT test to find particularly hearing threshold limits more quickly. In such cases 20Phon spacing is employed. Again, any granularity can be used but a 10 Phon base is a good compromise between accuracy, user-friendliness and speed.

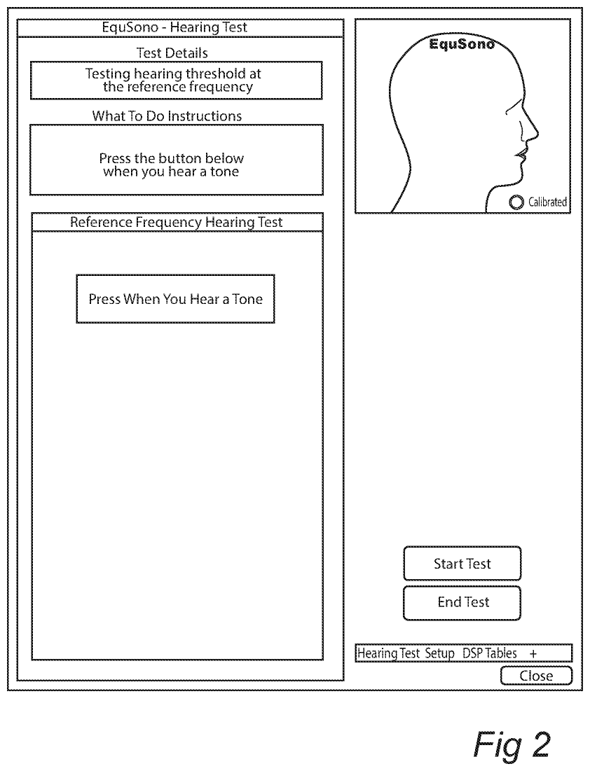

FIG. 2 shows a graphical user interface (GUI) from the ELHT software application. The figure displays Step 1 GUI with supporting functionality for setting up the test and generation of correction tables for a DSP hearing correction apparatus.

The lowest ELHT level is normally set to 10Phon if the test subject has low to no hearing loss at the selected reference frequency. If the test subject experience hearing loss at the selected reference frequency the lowest level can be increased.

The lowest ELHT level should be just above the test subject's hearing threshold at the reference frequency i.e., 0-10 Phon above. To assess hearing ability a minimum of one more higher sound pressure level is required. More sound pressure test levels offer a better assessment of the test subject's hearing ability. Normally four test levels with a sound level spacing of 10 Phon produces acceptable results. Testing at more levels obviously increase the burden put on the test subject to carry out the test but will provide a better understanding of hearing ability across a larger dynamic range.

### Step 1, Selection of Reference Frequencies

The aim of the reference selection step is to find a reference frequency for each ear where the hearing ability is as normal as possible without significant hearing loss. Each ear is tested individually to establish suitable frequencies, one for each ear. The reference frequency can be the same for both ears or, if required, it can be different for each ear.

It is desirable to locate the reference frequency as much as possible in the middle of the tested frequency range. It is easier to compare perceived loudness of two sounds if their frequency contents are closer. Age related hearing loss normally occur at higher frequencies with hearing ability in the lower frequency range remaining relatively intact. Hearing loss due to exposure to high sound levels also typically occur at higher frequencies. Therefore, it is advantageous to start the reference frequency selection test with an 800 Hz or 1200 Hz warble tone. 1200 Hz is more in the middle of the tested range than 800 Hz but some hearing loss is occasionally present at 1200 Hz which makes 800 Hz more viable in some cases. Eligible reference frequencies are 400 Hz, 800 Hz, 1200 Hz, 1600 Hz, 2400 Hz, 3200 Hz and 4800 Hz. In some cases, 200 Hz and 6800 Hz could be used but a lower accuracy is to be expected due to the increased distance between reference frequency and some of the test frequencies.

The higher frequencies in the eligible range can typically be useful for individuals suffering from what is popularly called cookie bite hearing loss, a loss of mid-range frequencies maintaining hearing at the lowest and highest frequencies. The lower frequencies can in general be used when there is significant loss at 1200 Hz, typically for persons suffering from severe hearing loss due to age or exposure to high sound levels.

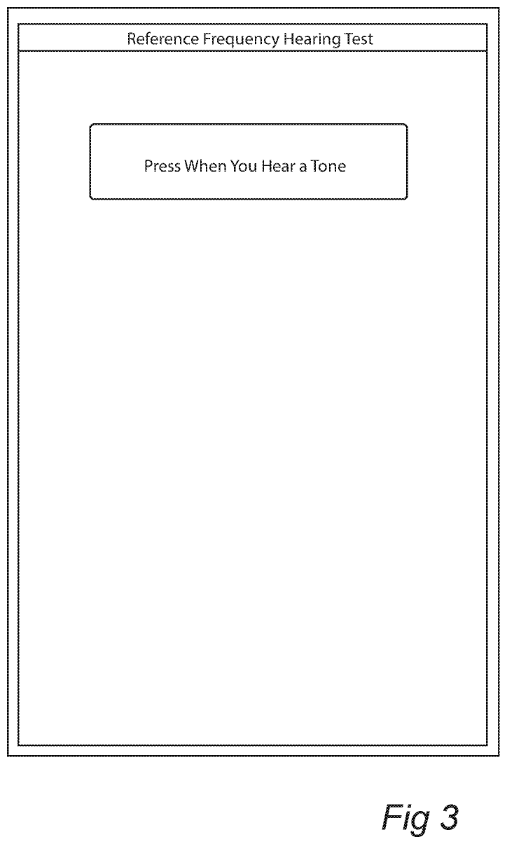

The following steps are performed for each ear individually, the tests are carried out using warble tones throughout. The reference frequency test start at 60 Phon and 1200 Hz. FIG. 3 shows the test panel GUI, the test subject should press the large button in the GUI panel whenever a sound is heard. The test frequency sequence is 1200 Hz, 800 Hz, 1600 Hz, 2400 Hz, 400 Hz and 3200 Hz.

If none of the sequence frequencies are heard at 60 Phon, 200 Hz and finally 6800 Hz are also tested at 60 Phon. If none are heard, the level is increased to 70 Phon and the sequence is retested. If none are heard, the hearing loss is too severe, the test is terminated.

When a frequency in the sequence is heard, stop at that frequency and test at 20 Phon below the current level. If heard, test at another 20 Phon below. Continue until 10 Phon is reached and heard, stop at that frequency and use it as reference frequency. For a test person with normal hearing at the test frequency the level sequence will be 60 Phon, 40 Phon, 20 Phon, 10 Phon and stop if the test tone is heard at all levels. If the hearing threshold is between 20 Phon and 30 Phon the sequence will be 60 Phon, 40 Phon, 20 Phon, 30 Phon and then stop. 30 Phon will be the lowest level at which the test frequency is heard.

If the lowest level at which the test frequency is heard is above 10 Phon, test the next frequency in the sequence starting at the lowest level for the current frequency.

When one of the following frequencies in the sequence is heard, test at that frequency at 10 Phon below the lowest level. Continue until 10 Phon is reached and heard, then stop at that frequency and use it as reference or stop earlier at the frequency that is heard at the lowest level and use that. If several frequencies are heard at a particular level use the first frequency in the sequence that can be heard as the reference.

The selected reference frequency/frequencies are used at all sound levels to balance the perceived loudness of the other test frequencies against i.e., one frequency per ear at all levels, which may be the same frequency for both ears, or not

### Step 2, Left and Right Ear Reference Frequency Level Balance

The reference frequencies, one for reach ear, that have been established in step 1 will be used as references to balance all other test frequency levels against. The perceived loudness of the other test frequencies should match the loudness of the references at all sound levels.

It is essential that the perceived loudness of the reference frequencies is the same in the left and right ear at all test levels. If they are not, the whole frequency range, in which loudness is balanced against the reference, will be skewed towards one of the ears. Further, if the loudness balance is skewed differently across levels the spatial sound field would be bouncing back and forth dependent on level.

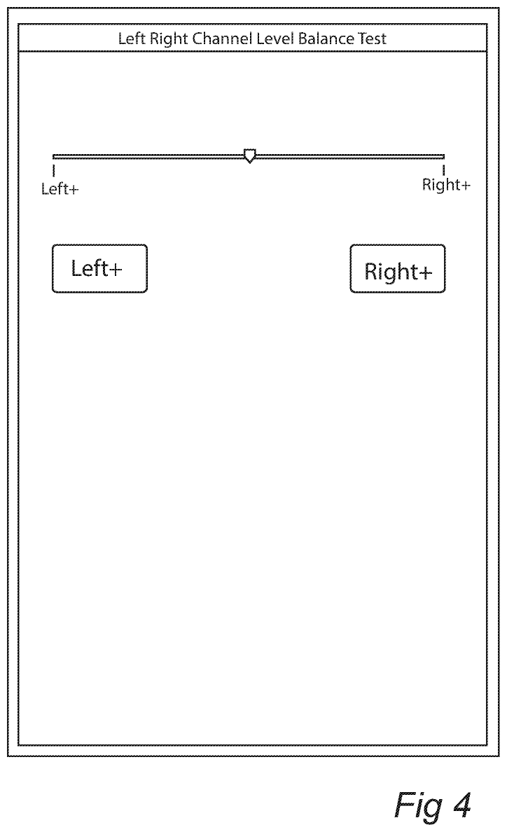

FIG. 4 shows the test GUI. The left and right balance is controlled by the slider and buttons on the panel. If the reference frequency is the same for left and right ear, inter ear balance is tested at each sound level that is going to be used in the test.

Only gain should be used to balance the perceived left right loudness level, not attenuation. The loudest frequency is the frequency where hearing ability is best and should consequently be used to balance loudness against i.e., apply gain to the softer sounding ear.

If the reference frequency is different for left and right ear, inter ear balance must be tested at both frequencies at each sound level that is going to be used in the test. As an example, 1200 Hz is used as reference frequency for the left ear and 800 Hz for the right ear. At first, the left ear loudness at 800 Hz is balanced against the left ear reference at 1200 Hz, so that the perceived loudness in the left ear is the same at both frequencies. Similarly, 1200 Hz is balanced against the reference 800 Hz in the right ear. Then 1200 Hz and 800 Hz in the right ear is balanced against the left ear reference of 1200 Hz and in the same way left ear is balanced against the 800 Hz reference of the right ear. Gain is applied to all frequencies in both ears so that the softer frequencies are balanced against the loudest frequency i.e., no gain applied to the loudest frequency irrespective of ear and all other frequencies irrespective of ear have gain applied.

### Step 3, Equal Loudness Test

During the equal loudness test the perceived loudness of each reference frequency is compared to the loudness of test frequencies at different sound levels. The loudness of the test frequencies is adjusted so it becomes equal to the reference frequencies.

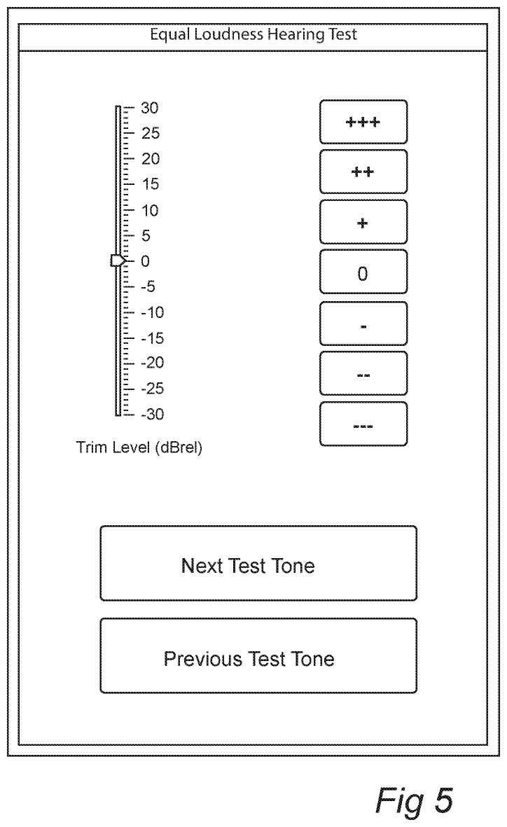

FIG. 5 shows the test panel GUI. The test frequency level is increased by pressing any of the plus buttons or decreased by pressing the minus buttons i.e., correction gain or loss is applied. The Next and Previous buttons jump forward or back to the next or previous test frequency and/or level.

The level range that is suitable for the equal loudness test is decided based on measured reference frequency hearing threshold data obtained in step 1. For a person with normal hearing or a moderate hearing loss, 70 Phon starts to get quite uncomfortable whereas 60 Phon is perceived as a high level but acceptable.

Therefore, it is not advisable to test at levels above 60 Phon under normal circumstances. 60 Phon is also a very safe level that will not cause any hearing damage. For test subjects with an established reference frequency hearing threshold below 40 Phon, 60 Phon is used as the start and highest test level. For test subjects with a hearing threshold of 40 Phon or 50 Phon at the reference frequency, 70 Phon is set as the start and maximum level. For test subjects with an elevated hearing threshold of 60 Phon or more at the reference frequency 80 Phon is set as the start and maximum level.

Preferably test frequencies should be 100 Hz, 200 Hz, 400 Hz, 800 Hz, 1200 Hz, 1600 Hz, 2400 Hz, 3200 Hz, 4800 Hz, 6800 Hz, 9600 Hz and 12800 Hz. Any frequency where excessive hearing loss is present might get discarded. Not unusually, the higher frequency test points 9600 Hz and 12800 Hz gets abandoned. Criteria for discarding a test frequency are when correction gain plus level exceeds safe limits of sound pressure or dynamic limits of the system. When an aggregate of correction gain plus level reaches above 90 dBspl it is considered unsafe, and it triggers a cancellation of further tests at that frequency at the current and higher sound levels. The equal loudness test starts at the highest sound level, normally 60 Phon. It uses 10 Phon level jumps as a minimum. If the 60 Phon test level shows a flat response i.e., no correction gain is required at any test frequency, 50 Phon is abandoned and the next test level is set to 40 Phon. All frequencies are tested again at the lower level. If gain is still not required at any test frequency, 30 Phon is jumped over and next test level is set to 20 Phon. If the 20 Phon test level shows a flat response, 10Phon is finally tested. If 20 Phon is not flat, 30 Phon is also tested before finally jumping to 10 Phon.

Generally, if correction gain is required at the current level the next level should be 10 Phon lower than the current. A 20 Phon step is only used if there is no correction gain applied at any test frequency. After a 20 Phon step down in level, if the response is not flat at the new lower level, next test should jump up 10 Phon in level before the level is finally lowered 10 Phon below current level.

In the first test series throughout all test frequencies and sound levels, the correction gain is initially set to 0 dB at each frequency and level and adjusted up and down at each frequency and level from the 0 dB starting point. After the first test series, having tested the selected test frequencies at appropriate sound levels, correction gain data is available for a second test run of selected test frequencies and sound levels. The newly obtained correction gain data is used as starting point for the second test series. For each following test series, the latest obtained correction gain values are used as starting points at each frequency and level, and this is repeated for all consecutive test series.

After the first test series and the second series it is possible to assess variability between first and second results. It has been found that if the correction gain differs significantly from the initial 0 dB it is more likely the obtained correction gain value is slightly wrong and therefore it is more important to repeat such test points to check variability. If variability is found to be high, use predominance for the closer to target starting points and calculate aggregate variability. A decision to stop is made either after a suitable maximum number of tests i.e., 5-10, or if the variability is not improving between tests anymore. Then, an average correction gain among lowest variability members in the test series is calculated. A suitable variability threshold is 3 dB.

Only the first and second test series contain all the selected test frequencies at all applicable sound levels. The third test series only include test frequencies at sound levels where variability is above the 3 dB threshold. Any subsequent test series similarly exclude test frequencies at sound levels where variability is below the 3 dB threshold, ultimately reaching an overall variability of less than 3 dB at all test points or reaching the maximum number of tests limit. An average correction gain among lowest variability members in the test series is calculated and used as the final gain correction.

Step 4, Phon to dBSPL Transposition

Sound pressure level measured in dBSPL is a logarithmic unit using a reference level of 20 micro pascals of sound pressure i.e., 0 dBSPL equals a physical sound pressure of 20 micro pascals. The ISO226 standard provides perceptual equal loudness information at different sound levels and frequencies. Perceptual levels are measured in Phon, which is a unit based on human perception of loudness. As an example, two tones, both at a level of 30 Phon, at the frequencies 1 kHz and 100 Hz respectively, will be perceived to be equally loud by a person with normal hearing ability. The tone at 100 Hz will however need to be at a higher physical sound pressure level compared to the tone at 1 KHz for them to be perceived as equally loud.

The equal loudness test's sound levels are based on the ISO226 standard and are measured in Phon. Since Phon is a perceptual level, each frequency test point and sound level must be translated from Phon to a technically derivable physical sound pressure measured in dBSPL.

The ISO226 standard only contains a finite number of numerical test points divided in frequency in a one third octave series from 20 Hz to 12 500 Hz. The number of numerical level test points are similarly limited.

The equal loudness test's chosen frequency points do not fully coincide with the ISO226 frequencies and the level data available in the ISO226 standard has a granularity that is too coarse for it to be useful directly. Consequently, the required data in between the numerical test points available in the standard must be interpolated. There are many possible mathematical interpolation methods that can be used, in this case the interpolated values are determined by cubic spline interpolation. Both level and frequency plane data must be interpolated to arrive at the required granularity.

During the equal loudness test, at each test frequency and sound level, the sound playback level is first translated from Phon to a physical sound pressure. Then the sound pressure level is changed from the initial physical sound pressure level until it is perceived to be equally loud as the reference frequency. The correction gain or loss that is added to the initial level is measured in logarithmic units, dB, and not in perceptual Phon.

As an example, we would like to test equal loudness at 100 Hz and 20 Phon. At first the corresponding physical sound pressure at 100 Hz must be calculated. Interpolated ISO226 data is used to derive the physical sound pressure level. In this case, 20 Phon at 100 Hz corresponds to approximately 48.4 dBSPL. Now, let us assume the level is perceived to be too low by the test subject and 10 dB positive correction gain is required to reach equal loudness. The test sound level must be 48.4 dBSPL+10 dB=58.4 dBSPL for equal loudness to be achieved. The 58.4 dBSPL level is however not equal to 30 Phon, the equation 20 Phon+10 dB=30 Phon is not correct. 20 Phon@100 Hz=48.4 dBSPL whereas 30 Phon@100 Hz=56.8 dBSPL, the 10 Phon difference at this specific frequency and level, only amounts to 8.4 dBSPL and not 10 dB. Test sound level is in Phon units, the correction gain is in dB, which is a basic logarithmic unit, not perceptual.

The transposition from Phon to dBSPL as reference for hearing correction gain should be done in this fourth step. Measured correction gain data is interpolated from Phon reference to dBSPL reference. Again, there are many possible mathematical interpolation methods that can be used, in this case interpolated dBSPL referenced values are determined by cubic spline interpolation.

Any real time signal processing employed to correct hearing loss will receive physical sound pressure information from microphone(s) and generate physical sound pressure with transducer(s). It is of course possible to make physical sound pressure to perceptual unit transformations back and forth in a real time application. It will however unavoidably require unnecessary additional calculation steps, which will both consume processing bandwidth and energy. Typically, energy is in very short supply in wearable earbuds, hearing aids or similar products. Calculations in real time signal processing are therefore preferably made referenced to physical sound pressures which constitutes and energy efficient approach to accomplish a desired hearing correction. Thus, correction gain data from the equal loudness test referenced to dBSPL levels should be available to a real time signal processing apparatus.

### Step 5, Post Processing of Measured Hearing Data

The data post processing step aims to extend measured hearing correction data to cover a dynamic range at least between 0 dBSPL to 110 dBSPL. Below 0 dBSPL the same correction gain as at 0 dBSPL will be used and at nearly all frequencies the level is below the hearing threshold anyway. Similarly, levels above 110 dBSPL have the same correction gain as at 110 dBSPL and such high levels will not be amplified in any case, they are attenuated by a maximum level limiter.

The equal loudness hearing test normally provides data from 10 Phon to 60 Phon in 10 Phon increments at each frequency. After the Phon to dBSPL transposition, data is available referenced to physical dBSPL units. Sometimes the available level range is smaller but a minimum of two and normally four to six level measurement points are available.

At first the level granularity is increased. Interpolation can be achieved mathematically in many ways, in this case cubic spline data interpolation is employed between the raw data points followed by a multiple order linear phase FIR average filtration of the interpolated data set. Filtration is made to smooth out local variations in the measured data to achieve an improved accuracy. The interpolation can generate as many datapoints over the available dynamic range as desired, in this case a 0.5 dB interval between data points is used.

Secondly, the dynamic range is extended below the lowest measured level down to 0 dBSPL. For this purpose, the lowest levels of the interpolated and filtered data are used. A straight-line, first order polynomial, derivative fit is made to the derivative of the lower level part of the interpolated and filtered data. The first order polynomial is then used to extend the data below the lowest measured level. Similarly, the interpolated and filtered data at the highest measured levels is used to perform a first order polynomial fit to the derivative of the highest level part of the interpolated and filtered data. The resulting first order polynomial can then be used to extend data points above the highest measured level. Finally, a multiple order linear phase FIR average filtration of the entire extended data set is made. Filtration smooths out the transitions between measured and extended data to improve accuracy. FIG. 6 displays a 3D diagram with measured and extended correction gain data, called a gain sail, at seven different frequencies.

Next step involves identifying candidate correction frequencies where correction gain could be applied. Frequencies where the equal loudness test shows that correction gain is not required will not be used and are discarded. Frequencies where the equal loudness measurement shows that hearing loss is too severe for correction to be applied are also discarded. The indicators being that excessive correction gain is required to restore hearing or simply that the test was terminated because the sound level was too loud. It is not unusual that the highest frequencies, 9600 Hz and 12800 Hz, pose problems for people with significant age related hearing loss, making it impossible for them to hear these frequencies. The remaining candidate frequencies are then further analyzed to find the appropriate correction frequencies. First, correction peaks are located within the candidate correction frequencies.

Peaks in this case means frequencies where the required correction gain is higher than the neighboring frequencies. When the peaks are identified, each peak's “weight” is calculated. A peak's weight is a multiplication of distance to next peak and the peak's correction gain. Then, the dominant peak with the largest weight is added to the selected frequencies. This procedure is iterated to find more peaks among the identified peaks and new peaks are consecutively added to the selected frequencies if the frequency distance to an existing peak among the selected frequencies is larger than a threshold. A suitable threshold is a factor of two away from an existing frequency. When all identified peaks have been investigated the procedure stops, the correction gain frequencies are now selected.

When appropriate frequencies are selected two optional steps are carried out. If the target hearing correction apparatus is known, it is possible to limit correction gain already in this step thereby removing or at least minimizing issues with acoustic feedback caused by excessive amplification. A maximum correction gain individual for each frequency that correlates with the hearing correction apparatus' acoustic feedback properties can beneficially be applied to the correction gain data in this stage.

The final step aims to reduce amplification at the highest sound pressure levels.

Normally, amplification is not applied at high sound pressure levels since it is not required for hearing correction. As an example, correction gain required to correct hearing loss for a test subject is displayed in FIG. 6. The correction does not incorporate amplification above approximately 80 dBSPL at any frequency although at 20 dBSPL upwards of 40 dB is required at several frequencies. The required correction gain displayed in FIG. 6 is very common, no gain is usually necessary at higher sound pressure levels even for individuals suffering from quite substantial hearing loss. In rare cases when correction gain is present at high sound pressure levels it is desirable to reduce the gain to avoid additional hearing damage. In this step correction gain is therefore gradually reduced to zero at levels above 90d BSPL so that input level plus gain never produces an output exceeding 100d BSPL. The two threshold levels 90d BSPL and 100d BSPL are selected to mitigate risk of additional hearing damage and can be adjusted to any other desired level.

### Step 6, Gain Sail Optimization

FIG. 6 displays a 3D diagram with gain sail data from a person facing cookie bite hearing loss. The gain sail shows the required correction gain (amplification) that is required at the frequencies 1-7 at sound pressure levels from 0 dBSPL to 120 dBSPL.

The frequencies in this case are 400 Hz, 800 Hz, 1200 Hz, 1600 Hz, 2400 Hz, 3200 Hz and 12800 Hz. The gain sail reveals the measured correction gain required to fully restore hearing for the test subject and is a collection of correction gain table data for each frequency. Looking at the gain sail, it is apparent that at higher sound pressure levels no correction gain is required, but at lower levels significant amplification is. Above approximately 80 dBSPL no amplification is necessary at any of the frequencies, whereas at 20 dBSPL upwards of 35 dB to 40 dB is needed at the middle frequencies to correct the measured hearing loss.

To restore hearing loss a real time signal processing apparatus must dynamically change filter gain at the 7 frequencies depending on the input sound pressure level at each of the frequencies. The real time signal processing apparatus must, in this example, comprise 7 band pass filters that predominantly pass each of the frequencies separately to 7 sound pressure level detectors, one for each frequency. The level detector outputs are then used to calculate the momentarily required gain of 7 peaking filters, again one filter for each frequency. The number of necessary frequencies varies from case to case and the 7 in this case is only an example. In some cases just one frequency is required but in most cases two to five is enough. In theory, all the available frequencies could be needed but it is unusual.

FIG. 8 shows frequency responses of 8 correction gain peaking filters with suitable bandwidths. Gain overlap between these filters can be seen in the diagram. Significant overlap occurs, especially in the middle frequencies that are relatively closely spaced. Close spacing is necessary to attain a correction that fits well with any measured hearing loss. The relatively wide bandwidth of the filters is also necessary, wide bandwidth filters have good time domain behavior whereas narrow bandwidth filters produce poor time domain response.

Good time domain behavior is required, human hearing is very sensitive to time domain behavior of sounds. If sound is appearing from drumming on wood or metal, a violin or trumpet being played is all interpreted by human hearing from the differences in time domain properties of the sound. A hearing restoration system obviously cannot be allowed to introduce time domain irregularities that would diminish sound quality making it harder to hear and interpret sound. Consequently, correction gain peaking filters must have wide bandwidths to maintain sound quality and they will for that reason always have overlapping responses as exemplified in FIG. 8.

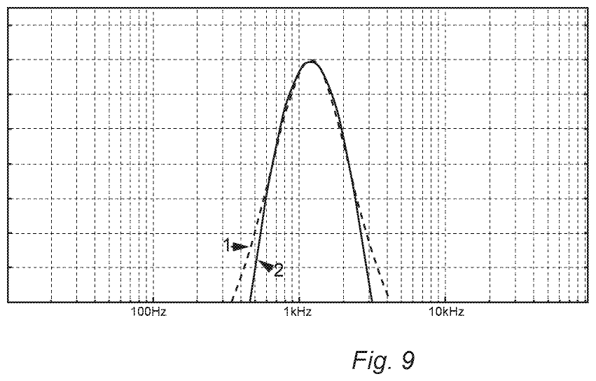

To achieve good tracking between dynamically applied correction gain and momentary sound pressure the correction peaking filter bandwidth and the level detector bandpass filter must have comparable frequency responses. FIG. 9 shows a correction peaking filter frequency response at 1200 Hz, trace 1, overlapping a suitable detector bandpass filter at 1200 Hz, trace 2. The bandpass filter cannot be too narrow as it would produce a poor time domain response and therefore not track the monetary sound pressure well. If the time domain tracking is bad, the measured momentary sound pressure would be incorrect and so would the applied correction gain that is calculated from the measured sound pressure. The bandpass filter bandwidth cannot be much wider than the correction peaking filter either as this would also produce incorrect sound pressure level measurements. With a wide band filter, sounds at frequencies far from the center frequency would be given too much weight in the measurement and gain would consequently be reduced at the center frequency. This is obviously wrong since the sounds are not present at the center frequency and the applied gain at the center frequency would become too low. The best results from a sound quality point of view are obtained when these two filters have similar frequency responses, as exemplified in FIG. 9.

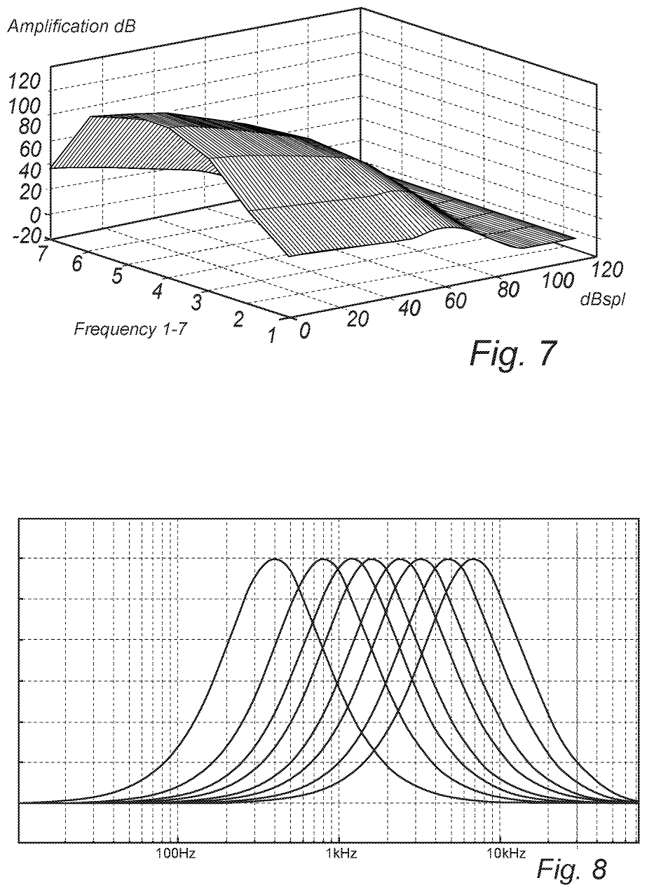

Employing filters with bandwidths shown in FIG. 8 to correct the measured hearing loss shown by the gain sail in FIG. 6 without taking gain overlap between the filters into account would produce far too much amplification. The gain sail in FIG. 7 displays total amplification without considering gain overlap. It is abundantly clear that the aggregate correction peaking filter amplification without factoring in gain overlap produces far too much gain, upward of +90 dB in the middle frequencies where it should only have been around +35 dB.

Whilst it would be possible to correct for over gain with a feedback correction network in a real time signal processing hearing correction apparatus, a feedback network will always produce time domain anomalies which are detrimental to sound quality. The severity of the problem with feedback becomes quite clear when one considers that with a low input sound level, say 20 dBSPL, the aggregate amplification rises to +90 dB. Then, as an example, when someone starts speaking and a significant input sound level arises from silence, the gain must instantly be turned down to a much lower level, looking at FIG. 6 probably around 25 dB. The gain then needs to change 65 dB within much less than 10 ms, which will cause significant distortion of the sound. However, not only will significant distortion be generated but the worst problem is gain miss tracking. The gain will initially be too high and sounds that are low in level will inadvertently be reproduced at a very high level causing severe initial transient overshoot. Soft sounds will sound as if they initially were almost like gunshots before the gain is turned down, which is obviously not satisfactory. With such large gain adjustments over short periods of time, gain tracking miss tracking will always be present. Feedback would also require additional real time processing steps that unavoidably consume processing bandwidth and energy.

There is, however, a better alternative to manage over gain which is termed gain sail optimization. With a detector bandpass filter bandwidth that matches the dynamic filter bandwidth it is possible to preprocess gain table data and optimize gain sail amplification, thereby avoiding significant distortion, gain miss tracking causing gunshot like issues and unnecessary real time calculations. Matching dynamic filter and detector band pass filter bandwidths are required for this to be possible so that the detector senses the sound level equal to how gain is applied by the dynamic filter. If the filter bandwidths are different preprocessing output will not be accurate.

Gain sail optimization is a mathematical problem that can be solved either using equations for an analytical solution or numerical iterative methods. Whilst an analytic solution is theoretically possible, given the complexity and very large number of variables and equations required would make such a solution wholly unfeasible. A numerical solution is therefore much preferred. ELHT utilizes a numerical iterative method to optimize the correction gain data.

Table data from the equal loudness hearing test, exemplified in FIG. 6, is used as target for the aggregate correction gain at each frequency and sound pressure level involved. The gain sail optimization aims to remove excess correction gain accumulated by adjacent frequency correction peaking filter boosts. The optimization is done through least squares numerical optimization of filter gain.

When the least squares optimization has found the best correction gains at all levels and frequencies, the adjusted values are saved for use in a real time signal processing apparatus.

Software functions, digital signal processing and algorithms can be implemented in many ways, from pure hardware implementations to pure software/firmware or a mix between the two. The DSP functions in the described invention use code written for a digital signal processor. The described ELHT and algorithms that produce input data to the hearing restoration apparatus is implemented in software running on a personal computer. The software could of course be implemented to run on any computing system such as a phone, tablet, or other device. It can also be implemented on a purpose-built target system resembling an audiometer for the new ELHT system as well as cloud computing resources.

### Specific Embodiments of the Present Invention

Below there are disclosed specific embodiments of the present invention. According to the present invention there is disclosed a software unit intended for hearing assessment and/or correction, said software unit being arranged for performing a method comprising the steps of:
performing a reference frequency hearing test to establish a reference frequency, for each ear of a test object; and
performing an equal loudness hearing test for each ear,
wherein the step of performing a reference frequency hearing test involves starting with a first frequency positioned within a given frequency range, providing a sound with a set sound level choosing a lowest test sound level in a range of from −10 to 50 Phon, to the test object to obtain a hearing response or not, changing the sound level with the first frequency, if the test object provides a hearing response at the chosen lowest test sound level in the range of from −10 to 50 Phon, then choosing the first frequency as the reference frequency, otherwise choosing another frequency within the given frequency range and repeating an iterative procedure with new frequencies within the given frequency range until the software unit detects a frequency X for which the test object provides a hearing response at the chosen lowest test sound level in the range of from −10 to 50 Phon or which is the frequency with the lowest sound level hearing response from the test object for the set frequencies to be tested, and then choosing that frequency X as the reference frequency.

According to one specific embodiment, the chosen lowest test sound level is chosen in the range of from 0 to 20 Phon, preferably in a range of from 5 to 15 Phon. As a suitable example, then chosen lowest test sound level is at or near 10 Phon.

According to one specific embodiment, the step of changing the sound level is performed by stepping down in sound level with the first frequency until there is no obtained hearing response from the test object.

Moreover, according to yet another embodiment, the software unit is programmed to choose a frequency closer to a middle of the given frequency range than a more peripheral frequency, preferably also to choose a lower frequency than a higher frequency when both are at an equal distance from the middle in the given frequency range.

Moreover, according to yet another embodiment, the software unit is programmed to choose new frequencies in the iteration according to a priority order. This may be performed by a set and implemented priority list.

Furthermore, according to yet another embodiment, the procedure of changing the sound level is performed with steps of at least 1 Phon for each step, preferably at least 5 Phon for each step, more preferably at least 10 Phon for each step, until reaching an absolute value of 10 Phon or a level for which the test object does not provide a hearing response. According to one embodiment, changing the sound level is performed by stepping down with steps of at least 1 Phon for each step, preferably at least 5 Phon for each step, more preferably at least 10 Phon for each step, until reaching an absolute value of 10 Phon or a level for which the test object does not provide a hearing response.

Moreover, according to one embodiment, the software unit is arranged for performing a left right channel level balance test comprising balancing perceived loudness of the reference frequencies between the left and right ear of the test object.

According to one embodiment, the step of balancing perceived loudness of the reference frequencies between the left and right ear of the test object being performed before the equal loudness hearing test for each ear.

According to yet another embodiment, the software unit is arranged for performing the left right channel level balance test between the steps of performing the reference frequency hearing test and performing the equal loudness hearing test. Furthermore, according to one embodiment, the software unit is arranged for performing a left right channel level balance test at all set measuring sound levels. Moreover, according to yet another embodiment, the step of performing an equal loudness hearing test for each ear is performed by comparing the perceived loudness, one frequency for each ear, of the reference frequency for each ear with a number of test frequencies.

Moreover, the software unit may be arranged for performing the equal loudness hearing test by providing different frequencies other than the set reference frequency and comparing responses from the test object at a number of appropriate sound pressure levels above a hearing threshold for the test object. According to yet another embodiment, the software unit is arranged for performing a first test series of the equal loudness hearing by providing a sequence of different sound levels to the test object and testing if correction gain is required at each sound level. Furthermore, according to yet another embodiment, the sequence of different sound levels in the first test series is provided by stepping down in sound level until a sound level is found where correction gain is required, preferably wherein a subsequent sound level being tested is higher than the sound level where correction gain was established to be required.

According to one embodiment, the software unit is arranged for performing a second test series where established correction gain values from the first test series is used for different sound levels provided to the test object to obtain new correction gain values, preferably wherein established correction gain values are used for a next test series iteratively. Moreover, the software unit may be arranged to assess variability of the correction gain values between different test series. Furthermore, a maximum threshold of the variability may be used as an input for a decision when stopping further testing for specific sound levels, preferably said maximum threshold is 3 dB. According to yet another embodiment, the software unit is arranged to calculate an average correction gain among lowest variable members in the test series performed and set as a final gain correction. Moreover, the software unit is suitably arranged for performing the equal loudness hearing test by using warble tones, preferably by using a range for warble tone modulation of from 1% to 10%, preferably with a modulation frequency in a range of from 5 Hz to 30 Hz.

As should be clear from the above, according to the present invention, the software unit is preferably also arranged for collecting obtained data from the test object. Furthermore, according to yet another embodiment, the software unit is arranged for discarding one or more tested frequencies, preferably based on hearing loss of the test object for said one or more tested frequencies.

Furthermore, according to one embodiment of the present invention, the software unit is arranged for setting a spl range for the equal loudness hearing test, suitably within a range of 10-70 Phon, and wherein the equal loudness hearing test is performed by changing the sound level, preferably at least 1 Phon each step, more preferably at least 5 Phon each step, more preferably at least 10 Phon each step. Moreover, according to one embodiment, the step of setting a spl range for the equal loudness hearing test is performed by stepping down from a high value within the set range.

According to one embodiment, the software unit is arranged in a computer unit or mobile device, such as a phone or tablet, or part of an embedded system, such as in a purpose-built product.

Moreover, according to yet another embodiment, the software unit or a connected data computing software is arranged for performing a transposition of obtained data from Phon to absolute dBSpl values by interpolating measured correction gain data from Phon reference to dBSpl reference. In relation to this, and as should be noted from the below, more or less any type of set-ups with different connected software units are possible according to the present invention. According to one embodiment of the present invention, the software unit or a connected data computing software is arranged for extending the dynamic range below the lowest measured level and/or above the highest measured level, preferably by using a first order polynomial, more preferably proceeded by a multiple order linear phase FIR average filtration. In this regard it should be noted that the application may be provided in a telephone or a computer unit, and then this may be connected to another program where additional calculation is being performed, such as in the cloud.

Furthermore, according to one embodiment, the software unit or a connected data computing software is arranged for identifying and selecting candidate correction frequencies where correction gain could be applied, by finding peak frequencies where required correction gain is higher than the neighboring frequencies, preferably then for calculating a weight of every identified peak based on a distance to a nearby located peak and the correction gain of the specific peak, more preferably then for identifying the dominant peak with the largest weight and adding the same to the selected frequencies.

Moreover, according to yet another embodiment, the software unit or a connected data computing software is arranged for performing a gain sail optimization involving a compensation for adjacent filter gain contributions where there is an aggregation of amplification from adjacent filters. The gain sail operation may be performed at different sites according to the present invention, such as in an additional software unit or in the main software. Moreover, according to one embodiment, the gain sail optimization involves removing excess correction gain accumulated by adjacent frequency correction peaking filter boosts, preferably by performing least squares numerical optimization of filter gain.

As hinted above, the software unit according to the present invention may be part of a system. In line with this, according to one embodiment, there is provided a system comprising a software unit according to the present invention and where the software unit comprises a digital signal processing (DSP) unit or is connected to another software unit or a device comprising a digital signal processing (DSP) unit, said digital signal processing (DSP) unit being arranged for processing and compensating collected obtained data from the test object.

According to one embodiment, the digital signal processing (DSP) unit involves one or more dynamic filters for compensating the data, preferably multiple dynamic filters are involved for compensating the data, preferably a number of from 2-20 dynamic filters are involved for compensating the data, preferably each dynamic filter is operating with dynamically changing gain dependent on dynamically changing input signal level at the filter frequency. Furthermore, according to yet another embodiment, the digital signal processing (DSP) unit is arranged for digital signal processing of amplification data and filter center frequencies, preferably by involving one or more filter blocks. Moreover, according to one embodiment, the digital signal processing (DSP) unit involves one or more dynamic filters for compensating the data, and wherein each dynamic filter involved has a center frequency in a range of 100 Hz- 12.8 kHz.

According to yet another embodiment, said one or more dynamic filters is operating with dynamically changing gain dependent on a dynamically changing input signal level at a certain filter frequency. Moreover, suitably said one or more filter blocks involved in the digital signal processing (DSP) unit comprise a band pass filter, a sound pressure detector, and a dynamic filter. Furthermore, according to yet another embodiment, the band pass filter is arranged to filter out and the detector is arranged to measure the signal level at the dynamic filter frequency and suppress sound signals present at other frequencies.

Moreover, according to one embodiment, the band pass filter has a low order, preferably second order, more preferably with Q below 1. Furthermore, according to yet another embodiment, the band pass filter and the dynamic filter, also seen as a peaking filter, has matching bandwidths.

Furthermore, according to one embodiment, said one or more filter blocks also comprise a gain table which uses table data, e.g. from algorithms, and translates the current input level to a gain setting in the dynamic filter.

Moreover, according to yet another embodiment, table amplification data and filter center frequencies are imported from the software algorithms to the digital signal processing (DSP) unit, and wherein the DSP unit comprises one or more filter blocks providing dynamically varying amplification, each handling an individual frequency range.

Furthermore, according to yet another embodiment, multiple filter blocks are used in the digital signal processing (DSP) unit.

1. A software unit intended for hearing assessment, said software unit being arranged for performing a method comprising the steps of:
performing a reference frequency hearing test to establish a reference frequency, for each ear of a test object; and
performing an equal loudness hearing test for each ear, wherein the step of performing a reference frequency hearing test involves starting with a first frequency positioned within a given frequency range, providing a sound, with a set sound level choosing a lowest test sound level in a range of from −10 to 50 Phon, to the test object to obtain a hearing response or not, changing the sound level with the first frequency, if the test object provides a hearing response at the chosen lowest test sound level in the range of from −10 to 50 Phon, then choosing the first frequency as the reference frequency, otherwise choosing another frequency within the given frequency range and repeating an iterative procedure with new frequencies within the given frequency range until the software unit detects a frequency X for which the test object provides a hearing response at the chosen lowest test sound level in the range of from −10 to 50 Phon or which is the frequency with the lowest sound level hearing response from the test object for the set frequencies to be tested, and then choosing that frequency X as the reference frequency.

2. The software unit according to claim 1, wherein the chosen lowest test sound level is chosen in the range of from 0 to 20 Phon, preferably in a range of from 5 to 15 Phon.

3. The software unit according to claim 1, wherein the step of changing the sound level is performed by stepping down in sound level with the first frequency until there is no obtained hearing response from the test object.

4. The software unit according to claim 1, wherein the software unit is programmed to choose a frequency closer to a middle of the given frequency range than a more peripheral frequency, preferably also to choose a lower frequency than a higher frequency when both are at an equal distance from the middle in the given frequency range.

5. The software unit according to claim 1, wherein the software unit is programmed to choose new frequencies in the iteration according to a priority order.

6. The software unit according to claim 1, wherein the procedure of changing the sound level is performed with steps of at least 1 Phon for each step, preferably at least 5 Phon for each step, more preferably at least 10 Phon for each step, until reaching an absolute value of 10 Phon or a level for which the test object does not provide a hearing response,
optionally changing the sound level is performed by stepping down with steps of at least 1 Phon for each step, preferably at least 5 Phon for each step, more preferably at least 10 Phon for each step, until reaching an absolute value of 10 Phon or a level for which the test object does not provide a hearing response.

7. (canceled)

8. The software unit according to claim 1, wherein the software unit is arranged for performing a left right channel level balance test comprising balancing perceived loudness of the reference frequencies between the left and right ear of the test object,
optionally the step of balancing perceived loudness of the reference frequencies between the left and right ear of the test object being performed before the equal loudness hearing test for each ear,
optionally the software unit is arranged for performing the left right channel level balance test between the steps of performing the reference frequency hearing test and performing the equal loudness hearing test,
optionally the software unit is arranged for performing a left right channel level balance test at all set measuring sound levels.

9. (canceled)

10. (canceled)

11. (canceled)

12. The software unit according to claim 1, wherein the step of performing an equal loudness hearing test for each ear is performed by comparing the perceived loudness, one frequency for each ear, of the reference frequency for each ear with a number of test frequencies.

13. The software unit according to claim 1, wherein the software unit is arranged for performing the equal loudness hearing test by providing different frequencies other than the set reference frequency and comparing responses from the test object at a number of appropriate sound pressure levels above a hearing threshold for the test object,
and/or the software unit is arranged for performing a first test series of the equal loudness hearing by providing a sequence of different sound levels to the test object and testing if correction gain is required at each sound level,
optionally the sequence of different sound levels in the first test series is provided by stepping down in sound level until a sound level is found where correction gain is required, preferably wherein a subsequent sound level being tested is higher than the sound level where correction gain was established to be required,
optionally the software unit is arranged for performing a second test series where established correction gain values from the first test series is used for different sound levels provided to the test object to obtain new correction gain values, preferably wherein established correction gain values are used for a next test series iteratively,
optionally the software unit is arranged to assess variability of the correction gain values between different test series,
optionally a maximum threshold of the variability is used as an input for a decision when stopping further testing for specific sound levels, preferably said maximum threshold is 3 dB,
optionally the software unit is arranged to calculate an average correction gain among lowest variable members in the test series performed and set as a final gain correction.

14. (canceled)

15. (canceled)

16. (canceled)

17. (canceled)

18. (canceled)

19. (canceled)

20. The software unit according to claim 1, wherein the software unit is arranged for performing the equal loudness hearing test by using warble tones, preferably by using a range for warble tone modulation of from 1% to 10%, preferably with a modulation frequency in a range of from 5 Hz to 30 Hz,
and/or wherein the software unit is arranged for collecting obtained data from the test object,
and/or wherein the software unit is arranged for discarding one or more tested frequencies, preferably based on hearing loss of the test object for said one or more tested frequencies.

21. (canceled)

22. (canceled)

23. The software unit according to claim 1, wherein the software unit is arranged for setting a spl range for the equal loudness hearing test, suitably within a range of 10-70 Phon, and wherein the equal loudness hearing test is performed by changing the sound level, preferably at least 1 Phon each step, more preferably at least 5 Phon each step, more preferably at least 10 Phon each step, optionally the step of setting a spl range for the equal loudness hearing test is performed by stepping down from a high value within the set range.

24. (canceled)

25. The software unit according to claim 1, wherein the software unit is arranged in a computer unit or mobile device, such as a phone or tablet, or part of an embedded system, such as in a purpose-built product,
and/or wherein the software unit or a connected data computing software is arranged for performing a transposition of obtained data from Phon to absolute dBSpl values by interpolating measured correction gain data from Phon reference to dBSpl reference,
optionally the software unit or a connected data computing software is arranged for extending the dynamic range below the lowest measured level and/or above the highest measured level, preferably by using a first order polynomial, more preferably proceeded by a multiple order linear phase FIR average filtration,
optionally the software unit or a connected data computing software is arranged for identifying and selecting candidate correction frequencies where correction gain could be applied, by finding peak frequencies where required correction gain is higher than the neighboring frequencies, preferably then for calculating a weight of every identified peak based on a distance to a nearby located peak and the correction gain of the specific peak, more preferably then for identifying the dominant peak with the largest weight and adding the same to the selected frequencies,
optionally the software unit or a connected data computing software is arranged for performing a gain sail optimization involving a compensation for adjacent filter gain contributions where there is an aggregation of amplification from adjacent filters,
optionally the gain sail optimization involves removing excess correction gain accumulated by adjacent frequency correction peaking filter boosts, preferably by performing least squares numerical optimization of filter gain.

26. (canceled)

27. (canceled)

28. (canceled)

29. (canceled)

30. (canceled)

31. A system comprising a software unit according to claim 1, wherein the software unit comprises a digital signal processing (DSP) unit or is connected to another software unit or a device comprising a digital signal processing (DSP) unit, said digital signal processing (DSP) unit being arranged for processing and compensating collected obtained data from the test object.

32. The system according to claim 31, wherein the digital signal processing (DSP) unit involves one or more dynamic filters for compensating the data, preferably multiple dynamic filters are involved for compensating the data, preferably a number of from 2-20 dynamic filters are involved for compensating the data, preferably each dynamic filter is operating with dynamically changing gain dependent on dynamically changing input signal level at the filter frequency,
and/or wherein the digital signal processing (DSP) unit is arranged for digital signal processing of amplification data and filter center frequencies, preferably by involving one or more filter blocks.

33. (canceled)

34. The system according to claim 32, wherein the digital signal processing (DSP) unit involves one or more dynamic filters for compensating the data, and wherein each dynamic filter involved has a center frequency in a range of 100 Hz-12.8 kHz,
and/or wherein said one or more dynamic filters is operating with dynamically changing gain dependent on a dynamically changing input signal level at a certain filter frequency.

35. (canceled)

36. The system according to claim 34, wherein said one or more filter blocks involved in the digital signal processing (DSP) unit comprise a band pass filter, a sound pressure detector, and a dynamic filter, optionally the band pass filter is arranged to filter out and the detector is arranged to measure the signal level at the dynamic filter frequency and suppress sound signals present at other frequencies.

37. (canceled)

38. The system according to claim 36, wherein the band pass filter has a low order, preferably second order, more preferably with Q below 1, and/or wherein the band pass filter and the dynamic filter has matching bandwidths.

39. (canceled)

40. The system according to claim 34, wherein said one or more filter blocks also comprise a gain table which uses table data, e.g. from algorithms, and translates the current input level to a gain setting in the dynamic filter.

41. The system according to claim 34, wherein table amplification data and filter center frequencies are imported from the software algorithms to the digital signal processing (DSP) unit, and wherein the DSP unit comprises one or more filter blocks providing dynamically varying amplification, each handling an individual frequency range.

42. The system according to claim 34, wherein multiple filter blocks are used in the digital signal processing (DSP) unit.
